(19) United States
(12) Patent Application Publication
(10) Pub. No.: US 2020/0030571 A1
(43) Pub. Date: Jan. 30, 2020

(54) Cyclical Visual Effect To Assist Meditation And Mindfulness

(71) Applicant: James Gilbert Esch Duran, Centennial, CO (US)

(72) Inventor: James Gilbert Esch Duran, Centennial, CO (US)

(21) Appl. No.: 16/521,551

(22) Filed: Jul. 24, 2019

Related U.S. Application Data
(60) Provisional application No. 62/703,328, filed on Jul. 25, 2018.

(51) Int. Cl. A61M 21/02

(52) U.S. Cl. CPC A61M 21/02; A61M 2021/005

(57) ABSTRACT

The present invention is a visual effect software that operates in a cyclical manner to guide breath meditation and promote mindfulness. The present invention works closely with a device operating system, allowing the user to run other applications in addition to the invention. This invention is differentiated in its ability to guide meditation and make the user more mindful alongside regular device use, without a user interface or information input. The invention is unique in its novel form of visual communication, communicating with users via overall pixel-quality time differentials, as opposed to absolute pixel states. The list of applicable devices is comprised of computers, smartphones, and tablets, and works with attached devices like projectors. After a user learns the breath meditation, the invention can make the user more mindful whenever the user perceives its presence. The effectiveness of the invention increases when the user completes complementary guided meditations.

### CROSS-REFERENCES TO RELATED APPLICATIONS

This application claims the benefit of U.S. Provisional Application Ser. No. 62/703,328 filed on Jul. 25, 2018 titled “Cyclical Visual Effect To Assist Meditation And Mindfulness” which is incorporated herein by reference in its entirety for all that is taught and disclosed therein.

### FIELD OF THE INVENTION

The present invention relates to meditation and mindfulness implemented during normal screen time on various electronic devices.

### BACKGROUND OF THE INVENTION

The stress and demands of modern life and the amount of time workers spend in front of screens can take a physical and mental toll upon individuals. Meditation and mindfulness can help alleviate the ramifications of these issues. Taking time out of a busy work day for meditation and mindfulness may not be possible for many individuals. Often the best time for a person to meditate is when they're stressed. However, stress often arises from being busy or feeling overwhelmed. Times like these are when people are least inclined to stop what they are doing. A way to incorporate meditation and mindfulness without a disruption of work time is therefore needed. If mindfulness techniques could be administered in tandem with screen activity, people can be more mindful without stopping what they are doing. Additionally, people could practice mindfulness techniques during screen-time in perpetuity, effectively increasing the amount of mindfulness training that people can fit into their schedule during the day.

### SUMMARY OF THE INVENTION

This Summary is provided to introduce in a simplified form a selection of concepts that are further described below in the Detailed Description. This Summary is not intended to identify key or essential features of the claimed subject matter, nor is it intended to be used to limit the scope of the claimed subject matter.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or class of elements, such as X1-Xm, Y1-Yn, and Z1-Zo, the phrase is intended to refer to a single element selected from X, Y, and Z, a combination of elements selected from the same class (e.g., X1 and X2) as well as a combination of elements selected from two or more classes (e.g., Y1 and Z3).

It is to be noted that the term “a entity” or “an entity” refers to one or more of that entity. As such, the terms “a” (or “an”), “one or more,” and “at least one” can be used interchangeably herein. It is also to be noted that the terms “comprising,” “including,” and “having” can be used interchangeably.

The term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof, shall include all those described in the summary of the invention, brief description of the drawings, detailed description, abstract, and claims themselves.

Unless the meaning is clearly to the contrary, all ranges set forth herein are deemed to be inclusive of the endpoints.

The present invention is a global visual effect software that operates in a cyclical manner to assist meditation and mindfulness. A global visual effect changes overall screen pixel quality like hue, saturation, lightness, brightness, dimness, opacity, or luminosity. This can be thought of as a screen overlay that changes in opacity, however the same affect can be accomplished through directly altering the pixel properties communicated to the screen. For this reason, cyclical changes of the visual effect are hereafter referred to as cyclical changes of the visual effect intensity.

The present invention works closely with a device operating system, allowing the user to view and operate other applications in tandem with the visual effect software. This invention is differentiated in its ability to guide meditation to make the user more mindful visually while the application is minimized or running in the background. The invention is unique in its novel form of visual communication. The invention communicates with users via changes in overall pixel quality, as opposed to absolute pixel states. This novel form of communication means that the present invention can be used in conjunction with other software applications. It can be used on any device having a display screen. The list of applicable devices includes, but is not limited to, computers, smartphones, and tablets. Common mindfulness techniques include breath meditation and awareness of the breath. Users learn to inhale as the visual effect intensity increases, exhale as the visual effect intensity decreases, and pause breathing as the visual effect remains at constant intensity.

After learning, the present invention can visually guide meditation through a cycle of visual effect intensity. Additionally, the cyclical visual effect can serve as a reminder of the breath to increase awareness of the breath, whether or not the user is breathing according to the cyclical visual effect.

### BRIEF DESCRIPTION OF THE DRAWINGS

FIG. 1 shows a block/schematic diagram of the learning method in an embodiment of the present invention.

FIG. 2 shows a block/schematic diagram of the cyclical relationship between changing visual effect intensity and user breathing in an embodiment of the present invention.

FIG. 3 shows a screen with a visual effect at a minimum intensity in an embodiment of the present invention.

FIG. 4 shows the screen with the visual effect at an intensity between the minimum intensity and a maximum intensity in an embodiment of the present invention.

FIG. 5 shows the screen with a visual effect at the maximum intensity in an embodiment of the present invention.

FIG. 6 shows a screen with the visual effect at an intensity between minimum intensity and maximum intensity, and a superimposed visual instruction to inhale in an embodiment of the present invention.

FIG. 7 shows a screen with the visual effect at an intensity between minimum intensity and maximum intensity, and a superimposed visual instruction to exhale in an embodiment of the present invention.

FIG. 8 shows the screen with the visual effect at a minimum intensity, and a superimposed image instructing the user to rest or pause breathing in an embodiment of the present invention.

FIG. 9 shows a screen capture of a video with the visual effect at an intensity between a minimum intensity and a maximum intensity, and a superimposed instruction to inhale as the visual effect increases in intensity in an embodiment of the present invention.

FIG. 10 shows a screen capture of a video with the visual effect at an intensity between the minimum intensity and the maximum intensity, and a superimposed instruction to exhale as the visual effect decreases in intensity in an embodiment of the present invention.

FIG. 11 shows a screen capture of a video with the visual effect at the minimum intensity, and a superimposed instruction to pause breathing in an embodiment of the present invention.

FIG. 12 shows a block/schematic diagram of guided meditations are used to make the user more mindful via a cyclical visual effect in an embodiment of the present invention.

FIG. 13 shows a block/schematic diagram of the visual effect cycle in an embodiment of the present invention.

FIG. 14 shows a block/schematic diagram of the invention application in an embodiment of the present invention.

FIG. 15 shows a block/schematic diagram of the overlay algorithm used in an embodiment of the present invention.

FIG. 16 shows a screen capture of a user interface in an embodiment of the present invention.

To assist in the understanding of the present disclosure the following list of components and associated numbering found in the drawings is provided herein:

### TABLE OF COMPONENTS

| Component | # |
|---|---|
| visual effect at a minimum intensity | 2 |
| visual effect intensity between a minimum | 4 |
| intensity and a maximum intensity | |
| visual effect at a maximum intensity | 6 |
| visual instruction to inhale | 8 |
| visual instruction to exhale | 10 |
| visual instruction to pause breathing | 12 |
| text | 14 |
| text | 16 |
| text | 18 |
| screen capture | 1600 |
| effect 1 button | 1602 |
| effect 2 button | 1604 |
| effect 3 button | 1606 |
| effect 4 button | 1608 |
| slider button | 1610 |
| sliding scale | 1612 |
| start/stop visual effect button | 1614 |
| video mediation button | 1616 |
| the toggle overlaid instruction button | 1618 |

### DETAILED DESCRIPTION

The invention may be implemented as a computer process, a computing system or as an article of manufacture such as a computer program product. The computer program product may be a computer storage medium readable by a computer system and encoding a computer program of instructions for executing a computer process.

The invention may also be practiced as a method, or more specifically as a method of operating a computer system. Such a system would include appropriate program means for executing the method of the invention. Also, an article of manufacture, such as a pre-recorded disk or other similar computer program product, for use with a data processing system, could include a storage medium and program means recorded thereon for directing the data processing system to facilitate the practice of the method of the invention. It will be understood that such apparatus and articles of manufacture also fall within the spirit and scope of the invention.

With the computing environment in mind, embodiments of the present invention are described with reference to logical operations being performed to implement processes embodying various embodiments of the present invention. These logical operations are implemented (1) as a sequence of computer implemented steps or program modules running on a computing system and/or (2) as interconnected machine logic circuits or circuit modules within the computing system. The implementation is a matter of choice dependent on the performance requirements of the computing system implementing the invention. Accordingly, the logical operations making up the embodiments of the present invention described herein are referred to variously as operations, structural devices, acts, applications, or modules. It will be recognized by one skilled in the art that these operations, structural devices, acts, applications, and modules may be implemented in software, firmware, special purpose digital logic, and any combination thereof without deviating from the spirit and scope of the present invention as recited within the claims attached hereto.

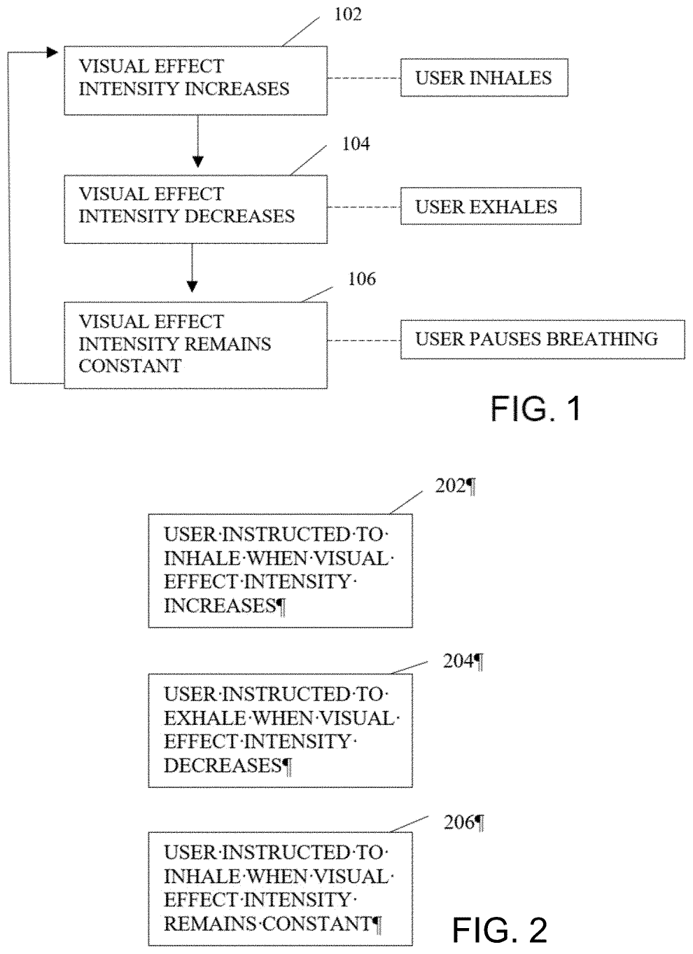

Referring now to the Figures, in which like reference numerals refer to structurally and/or functionally similar elements thereof, FIG. 1 shows a schematic/block diagram of the learning method in an embodiment of the present invention. Learning instruction described hereafter is visual, however audio instruction can be used in substitute of, or in combination with, video instruction.

Referring now to FIG. 1, the user is instructed to inhale in block 102 when the visual effect intensity increases. In block 104, the user is instructed to exhale when the visual effect intensity decreases. In block 106, the user is instructed to pause breathing when the visual effect remains at constant intensity. Hereafter, the present invention will be described according to the user inhaling as visual effect intensity increases, exhaling as the visual effect decreases in intensity, and pausing breathing as the visual effect remains at constant intensity. However, the user can alternatively be instructed to inhale as the visual effect decreases in visual intensity, exhale as the visual effect increases in intensity, and pause breathing when the visual effect remains at constant intensity. After the user learns to inhale as the visual effect increases, exhale as visual effect intensity decreases, and pause breathing as the visual effect remains constant, the user can do so without additional instruction. This means that after training the invention can guide user breathing without audio or visual instruction besides the visual effect intensity differential. This means that the invention can guide user breathing without interfering with information on the screen that is necessary to understand and interact with other applications. The user understands explicit visual screen communication from other applications through pixel-to-pixel relative perception. Since the invention alters overall pixel quality via a gradual, cyclical visual effect, the user maintains the ability to understand explicit visual communication on the screen via relative pixel perception. Since all pixels in the screen area are altered, the invention can communicate with the user no matter where they are looking on the screen. Consequently, the invention can visually communicate with the user while other applications communicate with the user. Therefore, the invention can operate without disrupting user activity necessary to use other applications such as word processors, spreadsheets, web browsers, games, or any other application.

FIG. 2 shows a schematic/block diagram of the cyclical relationship between changing visual effect intensity and user breathing in an embodiment of the present invention. Screens update constantly according to the frame rate. Visual effects are considered changing when the intensity of the visual effect of the current frame is different from the intensity of the visual effect from the previous frame. The visual effect may change at a rate lower than the maximum frame rate allowed by the screen. Referring now to FIG. 2, in block 202 the visual effect intensity increases for a period of time and the user inhales during the period of time. After the visual effect reaches maximum intensity, in block 204 the visual effect decreases for a period of time and the user exhales during the period of time. After the visual effect reaches minimum intensity, in block 206 the visual effect intensity remains constant for a period of time and the user pauses breathing during the period of time.

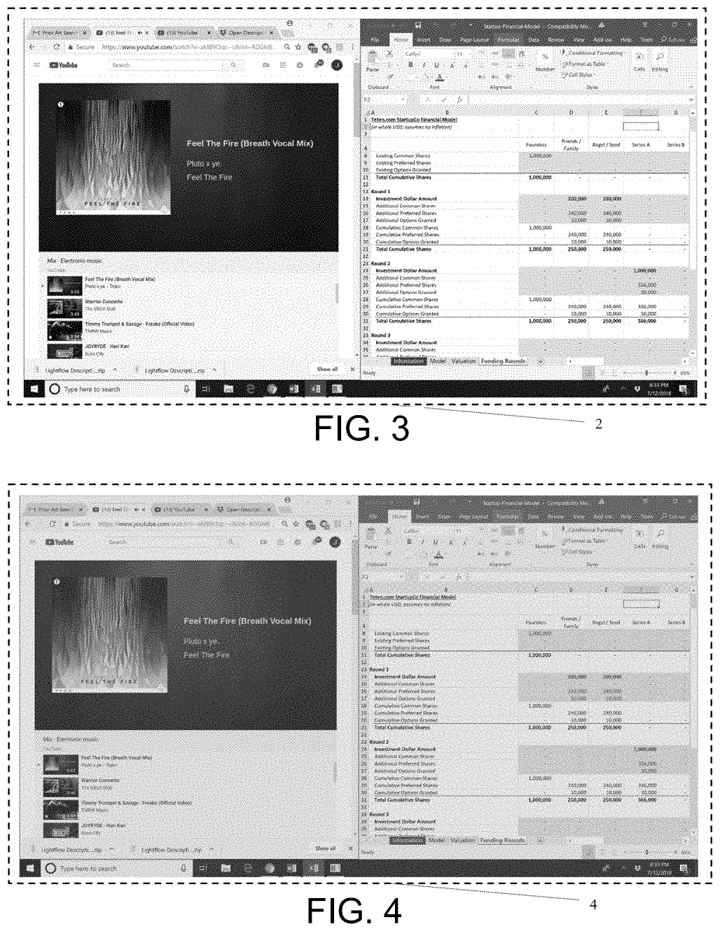

FIG. 3 shows a screen capture of the present invention with a visual effect at a minimum intensity 2 in an embodiment of the present invention. Referring now to FIG. 3, the screen capture shows two windows open on a display device associated with a laptop, table, desktop, or other comparable device. The left window is displaying a YouTube video, and the right window is displaying a spreadsheet. One skilled in the art will recognize that any one or more applications maybe displayed on the display associated with the associated device.

If the visual effect in the previously displayed frame is of a higher intensity, the user will start exhaling during the current frame. If the visual effect in the previously displayed frame is of a lower intensity, the user will start inhaling during the current frame. If the visual effect in the previously displayed frame is of an equal intensity, the user pauses breathing during the current frame.

FIG. 4 shows a screen capture of the present invention with the visual effect intensity between a minimum intensity and a maximum intensity 4 in an embodiment of the present invention. Referring now to FIG. 4, if the visual effect in the previously displayed frame is of a higher intensity, the user is exhaling. If the visual effect of the previously displayed frame is of a lower intensity, the user is inhaling.

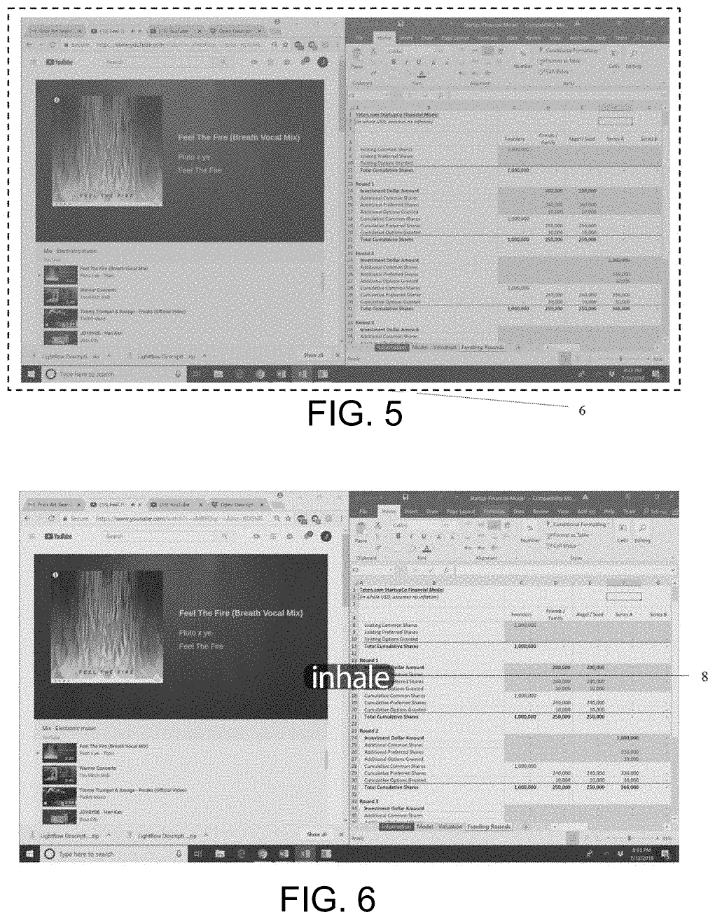

FIG. 5 shows a screen capture of the present invention with the visual effect at a maximum intensity 6 in an embodiment of the present invention. If the visual effect intensity in the previously displayed frame was lower, then the user will begin to inhale during the following frame. If the visual effect of the previously displayed frame was higher, then the user will begin to exhale during the following frame.

FIG. 6 shows a screen with the visual effect at an intensity between minimum intensity and maximum intensity, and a superimposed visual instruction to inhale 8 in an embodiment of the present invention. Referring now to FIG. 6, the user is inhaling as visual effect intensity is increasing. The user is also learning to inhale when the visual effect intensity increases via overlaid visual instruction to inhale 8 as the visual effect is increasing in intensity. The visual instruction to inhale 8 is overlaid over whatever screen information that would otherwise be present in that area of the screen. This method allows the user to use their device as normal while receiving additional visual instruction.

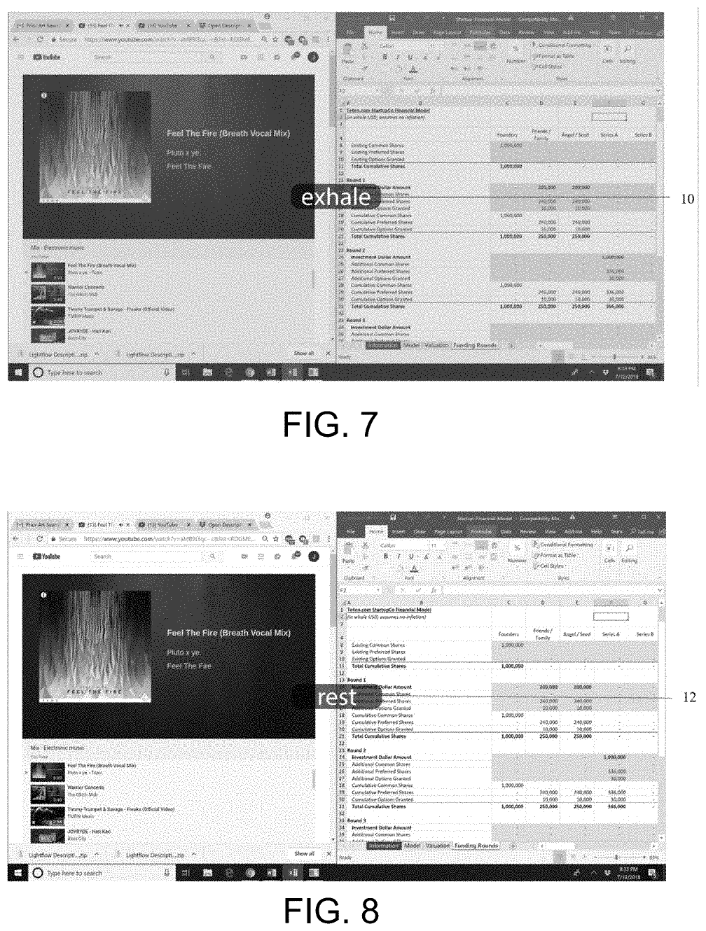

FIG. 7 shows a screen with the visual effect at an intensity between minimum intensity and maximum intensity, and a superimposed visual instruction to exhale 10 in an embodiment of the present invention. Referring now to FIG. 7, the user is exhaling as the visual effect intensity decreases. The user is also learning to exhale when the visual effect intensity decreases in intensity via the superimposed visual instruction to exhale 10 as the visual effect is decreasing in intensity. The visual instruction to exhale 10 is overlaid over whatever screen information that would otherwise be present in that area of the screen. This method allows the user to use their device as normal while receiving additional visual instruction.

FIG. 8 shows the screen with the visual effect at a minimum intensity, and a superimposed visual instruction to pause breathing 12 instructing the user to rest or pause breathing in an embodiment of the present invention. Referring now to FIG. 8, the user is pausing breathing as the visual effect intensity remains constant. The user is also learning to pause breathing when the visual effect intensity remains constant via the superimposed visual instruction to pause breathing 12 is overlaid over whatever screen information that would otherwise be present in that area of the screen. This method allows the user to use their device as normal while receiving additional visual instruction.

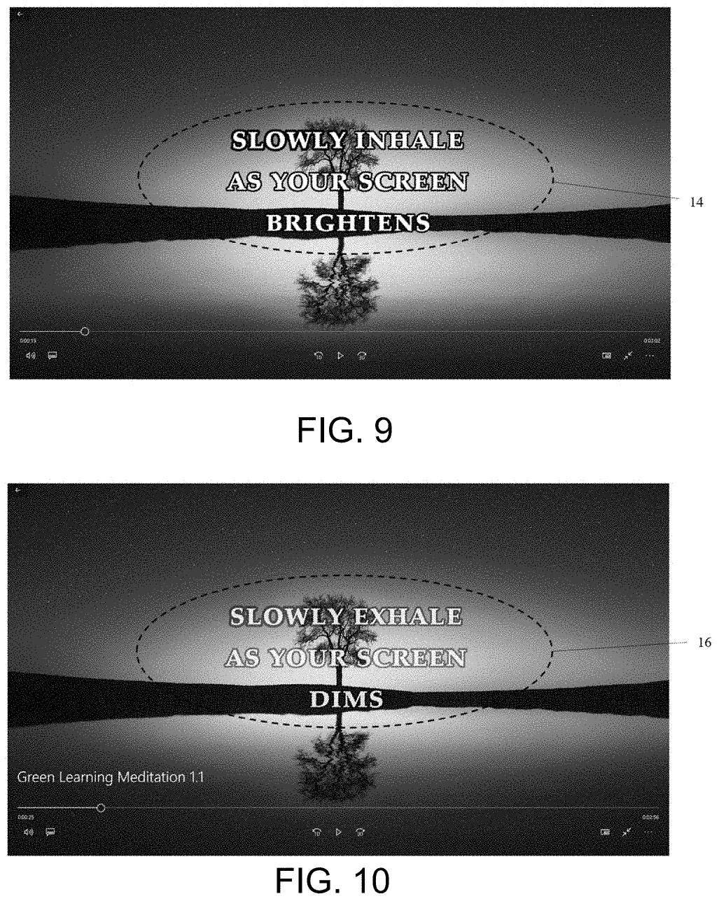

FIG. 9 shows a screen capture of a video with the visual effect at an intensity between a minimum intensity and a maximum intensity, and a superimposed instruction to inhale as the visual effect increases in intensity in an embodiment of the present invention. Referring now to FIG. 9, the user is instructed by text 14 displayed on the screen to inhale as the visual effect intensity increases. The text 14 may also be accompanied by an audio message to inhale as the visual effect intensity increases. In another embodiment, only the audio message is present and not text 14.

FIG. 10 shows a screen capture of a video with the visual effect at an intensity between the minimum intensity and the maximum intensity, and a superimposed instruction to exhale as the visual effect decreases in intensity in an embodiment of the present invention. Referring now to FIG. 10, the user is instructed by text 16 displayed on the screen to exhale as the visual effect intensity decreases. The text 16 may also be accompanied by an audio message to exhale as the visual effect intensity increases. In another embodiment, only the audio message is present and not text 16.

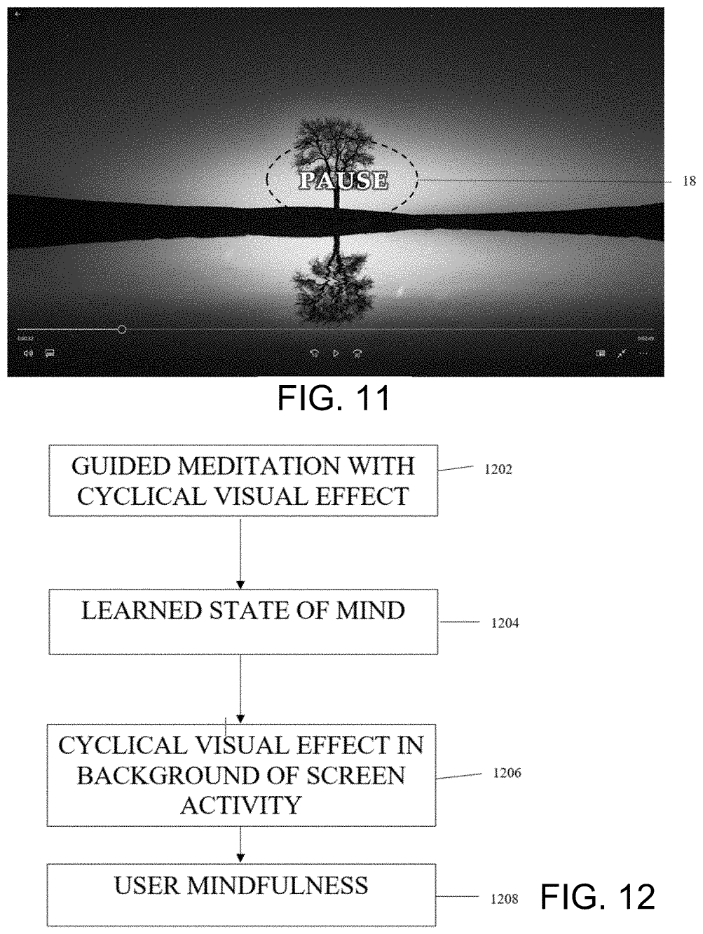

FIG. 11 shows a screen capture of a video with the visual effect at the minimum intensity, and a superimposed instruction to pause breathing in an embodiment of the present invention. Referring now to FIG. 11, the user is instructed by text 18 displayed on the screen to pause breathing as the visual effect intensity remains constant. The text 18 may also be accompanied by an audio message to pause breathing as the visual effect intensity increases. In another embodiment, only the audio message is present and not text 18.

FIG. 12 shows a block/schematic diagram of guided meditations are used to make the user more mindful via a cyclical visual effect in an embodiment of the present invention. Referring now to FIG. 12, in block 1202 users complete one or more guided meditations. Guided meditations are comprised of a cyclical visual effect, visual images, visual animations, overlaid images, overlaid videos, and/or audio.

Meditations are designed to promote user qualities or states of mind in block 1204 desirable to the user such as mindfulness, improved health outcomes, energy, productivity, focus, attention, performance on standardized tests, creativity, emotional regulation, overall psychological well-being, compassion, generosity, quality of life, calmness under stress, cognitive control, conflict monitoring, behavioral efficiency, calmness under stress, critical thinking, work performance, and reduced anxiety, depression, and emotional distress. Accordingly, the invention can then promote qualities like these in the background of other applications. This is done by promoting cognitive processes conducive to desirable user qualities or states of mind.

During guided meditations, the user learns behavior comprising states of mind and consciousness through a visual meditation that may or may not be complemented by audio. During a meditation, a cyclical visual effect in block 1206 is present. The user may or may not be encouraged to sync their breathing with the visual effect during the guided meditation.

Because the cyclical visual effect is present, the user associates the cognitive processes of the meditative state or behavior conducive to a desirable quality or state of mind with the cyclical visual effect resulting in user mindfulness in block 1208. This is accomplished by associating the top-down mental processes invoked by the guided meditation with the bottom-up perception of the cyclical visual effect. From that point forward, those top-down mental processes are encouraged whenever the user sees the cyclical visual effect. This process can also be described as associating the learned behavior from the meditation with the cyclical visual effect. From that point forward, the learned behavior is generated when the user sees the visual effect. This means that the user can attain that meditative state by merely perceiving the cyclical visual effect, without the need to synchronize breathing patterns communicated by the cyclical visual effect.

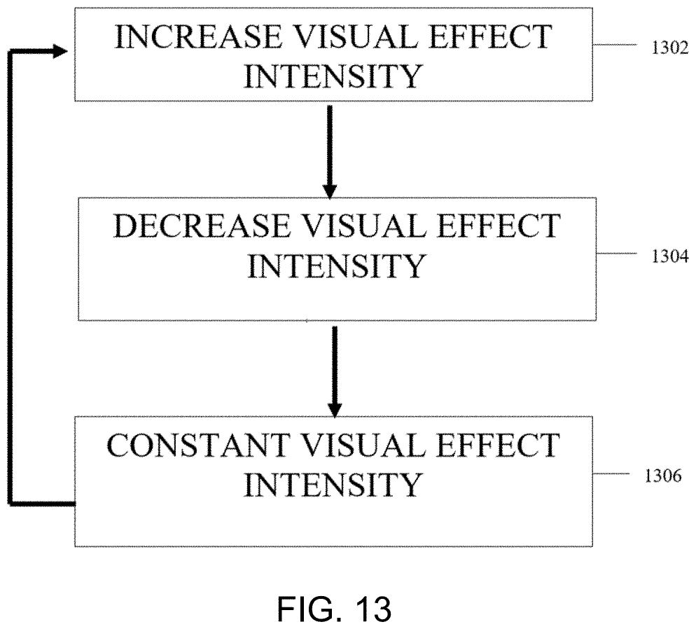

FIG. 13 shows a block/schematic diagram of the visual effect cycle in an embodiment of the present invention. Referring now to FIG. 13, the cycle consists of a period of time when the cyclical visual effect increases in intensity in block 1302, a period of time when the visual effect decreases in intensity in block 1304, and a period of time when the visual effect remains at constant intensity in block 1306. The summation of the durations of the visual effect increasing, decreasing, and remaining at constant intensity must total over one second. This is based on the idea that the presence of the cyclical visual effect reminds the user of the breath without the user having to breathe with the effect. These three periods of time can occur in any order. Whichever way the intensity periods of time are ordered, the intensity periods of time continue to cycle in that order repeatedly.

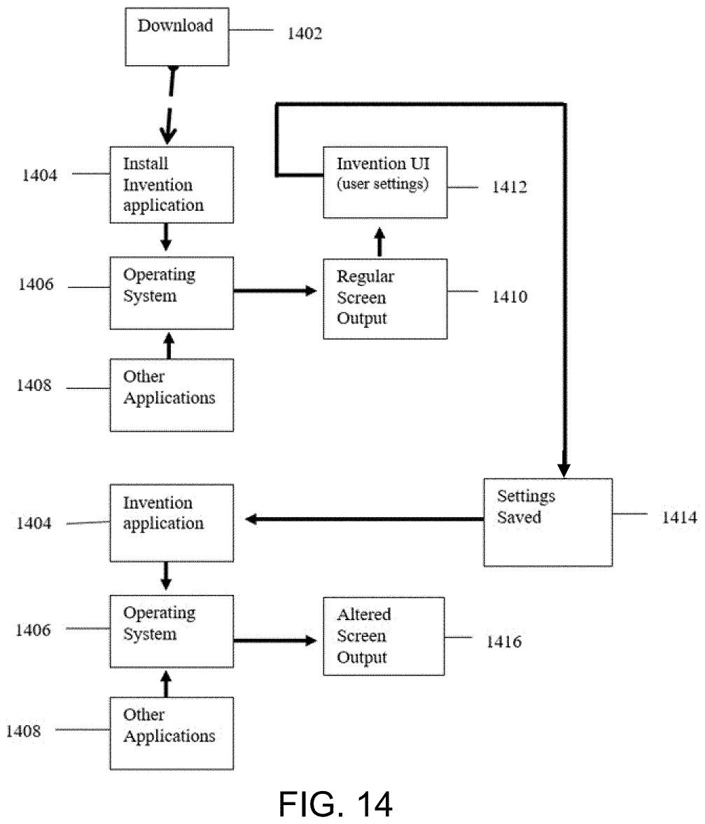

FIG. 14 shows a block/schematic diagram of the invention application in an embodiment of the present invention. The user in block 1402 downloads the invention application. In block 1404 the user installs the invention application on their device. In block 1406 the operating system can invoke the invention application and other applications from block 1408. In block 1410 the regular screen output is sent to a display. After installation, the user opens a visual user interface (see FIG. 6) in block 1412 to choose settings for the visual effect software. Settings can be a selection of visual effects and visual effect cycles with varying durations, minimum and maximum visual effect intensities, as well as the time duration for the periods of increasing intensity, decreasing intensity, and constant intensity which effectively change the inhale, exhale, and rest time of the cycle, the presence of overlaid instruction and the number of cycles for which overlaid instruction is present, and presets which will be saved in the software as default settings in block 1414. The user can also elect through the settings for the software to begin upon launch. This means that the invention will begin operating once the device is turned on without user input. Once selections have been made, the user interface is closed and the software begins to operate in block 1416 and the user can operate their device normally as the visual effect software displayed on a screen works in the background of other applications. When operating in the background, the invention application works by communicating with the operating system to alter screen pixel output. This means that other software can be used simultaneously with the invention application.

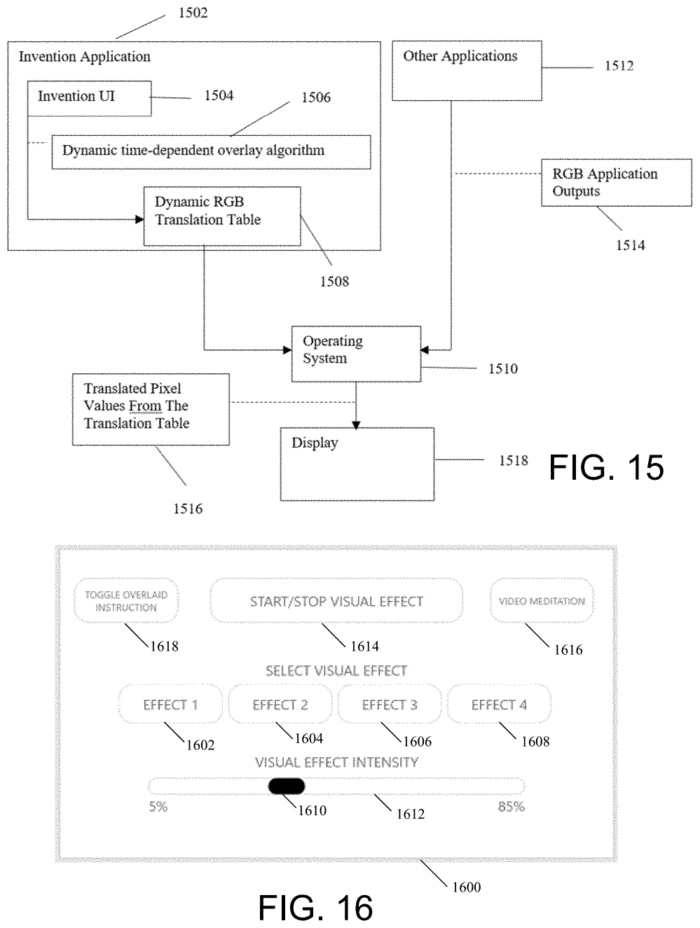

FIG. 15 shows a block/schematic diagram of the overlay algorithm used in an embodiment of the present invention. Referring now to FIG. 15, the invention application 1502 has the invention UI 1504 that receives the settings chosen by the user for the visual effects desired. The dynamic time-dependent overlay algorithm 1506 uses the saved visual effect RGB values, visual effect intensity, inhale duration, exhale duration, and rest duration and dynamically shifts the gamma color of a device display using a visual effect (determined by the RGB values) to create an oscillation between normal gamma and color shifted gamma at a frequency determined by the duration of the inhale, exhale, and rest cycle, and intensity determined by the visual strength setting. The dynamic time-dependent overlay algorithm dynamically constructs an RGB translation table 1508 every fraction of a second and passes this off to the device operating system 1510. Other applications 1512 pass their RGB application outputs 1514 to Operating system 1510. The translated pixel values from the translation table 1516 are then used for rendering on the display 1518.

FIG. 16 shows a screen capture 1600 of a user interface in an embodiment of the present invention. Referring now to FIG. 16, the user can select from four visual effects in one embodiment. Effect 1 button 1602, effect 2 button 1604, effect 3 button 1606, and effect 4 button 1608 each have a unique RGB value that is used to construct the translation table. The RGB values are preset for each of the effect buttons, but they each can be adjusted by the user as desired. The user can use a mouse or pointer to drag the slider button 1610 on the sliding scale 1612 to choose a visual effect intensity from 5% to 85% in one embodiment. The user can toggle on and off the visual effect by clicking on the start/stop visual effect button 1614. The user can select to view a video meditation session by clicking on the video mediation button 1616. The user can click on the toggle overlaid instruction button 1618 to overlay breathing instructions, syncing with the visual effect cycle.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. It will be understood by those skilled in the art that many changes in construction and circuitry and widely differing embodiments and applications will suggest themselves without departing from the scope of the disclosed subject matter.

What is claimed is:

1. A method for guiding breath meditation, the method comprising the steps of:
(a) instructing a user to inhale when a visual effect displayed on a screen changes in intensity in a first manner for a first period of time;
(b) instructing the user to exhale when the visual effect displayed on the screen changes in intensity in a second manner for a second period of time; and
(c) instructing the user to pause breathing when the visual effect intensity remains constant for a third period of time.
